(19) United States
(12) Patent Application Publication
(10) Pub. No.: US 2014/0330270 A1
(43) Pub. Date: Nov. 6, 2014

(54) METHOD OF ABLATING SCAR TISSUE TO ORIENT ELECTRICAL CURRENT FLOW

(71) Applicant: William J. Anderson, Ann Arbor, MI (US)

(72) Inventor: William J. Anderson, Ann Arbor, MI (US)

(21) Appl. No.: 14/256,132

(22) Filed: Apr. 18, 2014

Related U.S. Application Data
(60) Provisional application No. 61/832,485, filed on Jun. 7, 2013.
(60) Provisional application No. 61/819,194, filed on May 3, 2013.

(51) Int. Cl. A61B 18/14

(52) U.S. Cl. CPC A61B 18/14; A61B 2018/00577
USPC 606/41

(57) ABSTRACT

Methods of tissue ablation avoid undesirable electrical current patterns in the heart, for example. A method of ablating scar tissue to improve the circulation of electrical current comprises receiving information relating to the flow of electrical current across a surface of biological tissue, the flow defining an upstream direction and a downstream direction. In accordance with the invention, the biological tissue is ablated to form or modify a bluff body to create a tail portion, the tail portion being aligned so as to lie on the downstream side of the bluff body and pointing away from the direction of the incoming current. The method may include the step of forming or modifying the bluff body to create a symmetrical teardrop shape. The surface of biological tissue may be a three-dimensional surface, in which case the method may include the step of mapping the surface in two dimensions to orient the bluff body.

### REFERENCE TO RELATED APPLICATION

This application claims priority from U.S. Provisional Patent Application Ser. Nos. 61/819,194, filed May 3, 2013 and 61/832,485, filed Jun. 7, 2013, the entire content of both of which is incorporated herein by reference.

### FIELD OF THE INVENTION

This invention relates generally to tissue ablation and, in particular, to methods of tissue ablation to avoid undesirable electrical current patterns in the heart, for example.

### BACKGROUND OF THE INVENTION

For a traveling wave of electrical action, such as the electrical signals in the human heart, the definition of current direction is the direction of the propagation of the traveling wave. There is little doubt as to the meaning of direction of heart signals, and they are measured as traveling in the range of 0.1 to 1.0 meters/second. Charts of the propagation of the signal are available for the interior of the human heart.

It is known that scar tissue in the heart can affect the current flow. A common practice is to ablate (cauterize) this scar tissue to make it nonconducting. However, when a scarred area in the inner wall of the heart is ablated, the scar tissue is cauterized in a way as to act as an insulator to electrical flow. If, as is typical, this patch of cauterized tissue assumes the shape of a rough unstreamlined body (i.e., a bluff body), it will cease to transmit electrical signals within the patch, but can act as a barrier to the smooth electrical flow around it. Such scar tissue may assume roughly circular area or other shapes, depending upon condition and procedure.

Following a heart ablation ablation procedure, various conditions may arise, including ventricular tachycardia, manifest as a rapid heartbeat that starts in the ventricles. The conclusion is that in spite of ablation of a “bluff body,” there are circulating currents around the patch that can cause tachycardia or fibrillation.

### SUMMARY OF THE INVENTION

This invention relates generally to tissue ablation and, in particular, to methods of tissue ablation to avoid undesirable electrical current patterns in the heart, for example. A method of ablating scar tissue to improve the circulation of electrical current comprises receiving information relating to the flow of electrical current across a surface of biological tissue, the flow defining an upstream direction and a downstream direction. In accordance with the invention, the biological tissue is ablated to form or modify a bluff body to create a tail portion, the tail portion being aligned so as to lie on the downstream side of the bluff body and pointing away from the direction of the incoming current.

The preferred embodiment includes the step of forming or modifying the bluff body to create a symmetrical teardrop shape. The surface of biological tissue may be a three-dimensional surface, in which case the method may include the step of mapping the surface in two dimensions to orient the bluff body. The surface of biological tissue may comprise the inner surface of a human heart, and a thin, triangular heating device may be used to form and/or modify the bluff body.

### BRIEF DESCRIPTION OF THE DRAWINGS

FIG. 1 illustrates how Laplace equation describes the fluid flow around an airfoil;

FIGS. 2A-2B show a bluff body in 2-D potential flow; and

FIG. 3 depicts electrical current flow around a bluff ablated area.

### DETAILED DESCRIPTION OF THE INVENTION

In broad and general terms, this invention applies the extensive practice in aeronautics in designing bodies with known circulating fluid currents to the ablation of scar tissue to achieve optimal electrical circulation particularly within the heart. In particular, this inventor has recognized that the Kutta condition, the generation of fluid lift by circulation, and the sensitivity to boundary conditions all dictate that a bluff scar tissue area should be given a downstream sharp edge in order to control the amount of circulation to be a fixed, minimum value. This goal is achieved when the tail is aligned downstream with the dominant current direction. It is also desired to ablate the scar tissue in a symmetric pattern about the mean current direction to minimize circulating currents. That is, along with the sharp or pointed tail, this symmetry results in a “teardrop shape” and could cause zero circulating current. (This symmetry condition is called “zero camber” in airfoil theory.)

Contemporary devices using radio frequencies typically ablate a small circular area which is problematic. The ablation of a bluff body to give it a sharp tail will benefit from an ablation tool that can prepare a sharp corner. A thin, triangular heating device will suffice.

The invention is based on a “steady” interpretation of electrical current, and emphasizes the spatial part of the problem. When one looks at the actual heart signal, it is a traveling wave and has characteristics more like alternating current. This is due to the depolarization and repolarization of the heart's cells, which individually experience an alternating current. Since the transmission of such a signal is complicated, the invention implies a “separation of variables” which allows a separate discussion of the temporal and spatial problem. This approach is often used in studies of stability of systems and often simplifies the problem so that it can be solved.

In the heart electrical conduction problem, there are rotating electric fields called “rotors.” If one looks at the passage of a “mother” rotor over a barrier such as a flat plate insulator (obstacle), the mother rotor breaks into several rotors downstream of the barrier. This also happens in fluid flow, and supports the analogy with fluid flow used in this invention. The relation is imprecise because the viscosity of the fluid is important in the formation of downstream vortices, such as the Karman vortex street, and is not considered here.

### Definitions

Two-Dimensional: A domain of interest that involves only two coordinate directions. This could be a flat plane or a surface that can be mapped onto a flat plane.

Bluff Body: An impediment to electrical current flow or a fluid flow which has size perpendicular to the flow of the same order of magnitude as the size in the flow direction. Examples are a circle and a kidney-shaped region. Such regions are often created by the ablation of scar tissue in on the interior wall of the heart. Even after ablation, these ablated regions can continue to interfere with flow of electrical current.

Potential Function in Two Dimensions: A variable φ (x,y) which yields information about electrical currents or fluid velocities. Electrical current densities Iₓ and I_{y} are found by taking derivatives of the potential function:

$${I}_{x} = - σ \frac{\partial φ ( x , y )}{\partial x} {I}_{y} = - σ \frac{\partial φ ( x , y )}{\partial y}$$

where σ is the conductance.

Two-dimensional fluid velocities are also found by taking derivatives of a potential function:

$${v}_{x} = \frac{\partial φ ( x , y )}{\partial x} {v}_{y} = \frac{\partial φ ( x , y )}{\partial y}$$

LaPlace's Equation: A mathematical formula that can be used to describe electrical current or fluid flow behavior. We are interested in the specific application to two-dimensional electrical current in a uniform conductor, and two-dimensional fluid flow which is incompressible, inviscid and irrotational. The equation is:

$$\frac{{\partial }^{2} φ}{\partial {x}^{2}} + \frac{{\partial }^{2} φ}{\partial {y}^{2}} = 0$$

where the φ (x,y) can stand for either an electrical potential or a fluid potential.

Circulation: If one integrates the fluid velocity around a closed path according to the vector law,

Γ=∫_{c}\vec{v}·\vec{dl}

one obtains the “circulation” Γ of the flow. Although this will be zero about paths in a simply-connected region, it can be nonzero in a multiply-connected region. This means the circulation about an airfoil or a bluff body can be nonzero. This circulation is often called vorticity, and the lift on an airfoil is proportional to the amount of vorticity.

L=ρVΓ (Kutta-Joukowski Theorem, Ref 1)

Irrotational Flow: Inviscid, incompressible low-speed fluid flows have a local property called irrotationality, where in a simply connected region, no circulation can occur. This does not prevent circulation in the external region about a body in 2-D flow, however, and that circulation is the basis for the lift calculation for thin airfoils.

Kutta Condition: The requirement that fluid flow pass smoothly from the trailing edge of a slender airfoil. If the flow were required to turn the sharp corner, it would lead to infinite velocities at that point.

It is well known in aeronautics that it is important to have a sharp tail on the back of a subsonic lifting body. Thin aircraft wings have been designed that way for over one hundred years. The sharp tail causes the flow to pass smoothly over the trailing edge. The same equation (Laplace's Equation) governs both 2-D fluid flow and 2-D electrical flow. This invention recognizes that the same need for a sharp trailing edge holds for electrical current around 2-D ablated regions of the heart (bluff bodies).

In accordance with the invention, an ablated area should be fitted with a sharp tail so as to prevent unwanted circulation of electrical currents around the ablated area. This is done by adding a sharply pointed region behind the bluff body, aligned so as to lie on the downstream side of the bluff body, pointing directly away from the direction of the incoming current. This will cause the least amount of circulating current around the scarred area. Such a configuration will also fix the amount of circulation and make it less sensitive to material and other irregularities.

If a sharp tail is not properly added to the ablated area, the electrical current will have substantial amounts of circulating current. Furthermore, the amount of this unwanted circulating current will be extremely sensitive to details of the surrounding electrical field (mathematically described as boundary conditions).

### Mathematical Model of 2-D Fluid Flow Around Bodies

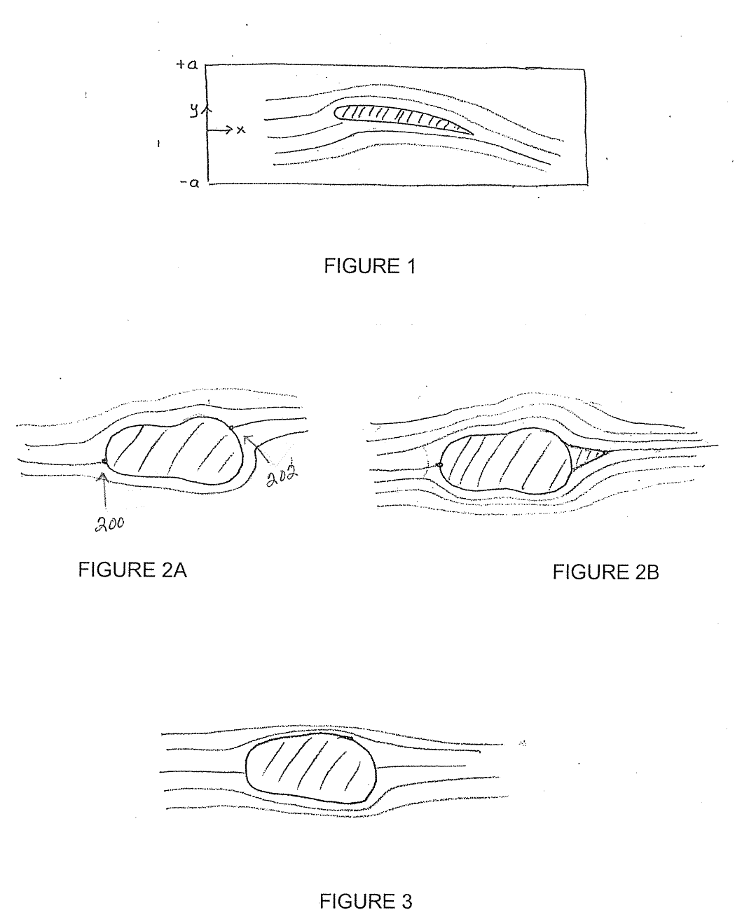

This invention results from “dissimilar modeling,” wherein an electrical current and a fluid flow are described by the same equation and have the same general characteristics (Ref. 2, 3). We will look at the fluid problem first. The Laplace equation is used to describe the fluid flow around an airfoil (FIG. 1). A rectangular region is used to bound the airfoil in this model.

The airflow is assumed to have an initial value vₓ(0,y)=V at the left boundary. At the upper and lower boundaries, there is no flow normal to the wall v_{y}(x,+a)=0 and v_{y}(x,−a)=0 The boundary condition at the right end is best moved to x=∞ and a pressure set to static pressure of the upstream (left boundary). Kutta and Joukowski calculated the lift generated on a specific type of airfoil and found it related to the “circulation” around the airfoil. This circulation leads to lift of:

L=ρVΓ (Kutta-Joukowski Theorem, Ref. 1)

The circulation has been fixed at a specific value due to the Kutta condition and depends on the presence of the sharp trailing edge of the airfoil.

If a bluff body is not given a sharp tail, experiments show that the flow lines around the body create circulation (FIGS. 2A-2B), and the amount of circulation is very sensitive to boundary conditions--to the point of being a problem of a “singular” nature. That is, the circulation can be made to take any value depending on the boundary conditions imposed. Experiments show that there is an upstream stagnation point 200 and a downstream stagnation point 202, as shown in FIG. 2A. The location of these points are extremely sensitive to the boundary conditions of the problem. As a result, most good information about lift and drag on bluff bodies is found by experiment rather than theory.

An interesting side issue is that there is no aerodynamic drag on bluff bodies in potential flow. This nonintuitive result is called D'Alembert's paradox. No comparable paradox exists for lift, however, and therefore there is circulating flow (and lift) around a bluff body.

It must be realized that the real case of fluid flow around bluff bodies is very complicated due to viscosity in the fluid. The potential theory is of little use in establishing the actual location of the stagnation points and of the circulation around a bluff body in a real viscous fluid flow.

### Mathematical Model of Electrical “Flow”

The electrical field in the heart is pulsating, according to the heart beat rate. The heart is contoured, and the current tends to travel on the inner surface, which can be mapped onto a 2-D space. It is useful to think of the steady-state current flow as a first approximation to current in this pulsating, curved surface.

The invention is useful when the dominant current direction is known. (There are areas of the ventricles where this would be so, as well as smaller portions of the atria and pulmonary vein.) If the dominant direction of the current is not known, or if the direction of the current changes markedly in time, then it may not be appropriate to ablate a more “aerodynamic” patch. The historical reasoning often used is that trees subjected to winds from all directions tend to have a trunk with circular cross section, and this is indeed optimal when the direction of the current is not known or is changeable.

A 2-D electrical potential can be assumed, with the incoming current from the left boundary and the current passing over an ablated area (FIG. 3).

The field equation is:

$$\frac{{\partial }^{2} φ ( x , y )}{\partial {x}^{2}} + \frac{{\partial }^{2} φ ( x , y )}{\partial {y}^{2}} = 0$$

with current densities defined as:

$${I}_{x} = - σ \frac{\partial φ ( x , y )}{\partial x} {I}_{y} = - σ \frac{\partial φ ( x , y )}{\partial y}$$

Boundary conditions can be applied on the sides of the “box” under consideration. The results of this calculation will be exactly the same as the case for fluid flow. There will be circulation of electrical current around the bluff body. Electrical engineers are typically not interested in the electrical equivalent of lift, drag and circulation, however, as those electrical quantities are not physical quantities of interest. On the other hand, we may appropriate the results from fluid flow calculations and see that the Kutta condition, the Kutta-Joukowski formula and the D'Alembert paradox all apply in the electrical conduction case. The conclusion is the same as in fluid flow—one should use a sharp trailing edge on a bluff body (FIG. 2B) immersed in an electrical field of two dimensions.

Future technology involving ablation of tissue through the thickness of the heart wall should also consider the use of this sharp trailing edge and a symmetric ablated shape. Those of skill in the art will appreciate that the expansion of the concepts described herein from 2-D to 3-D will be the same as the aerodynamic progression from airfoil theory to wing theory, such that the Kutta condition and zero camber advantages still apply.

1. A method of ablating scar tissue to improve the circulation of electrical current, comprising the steps of:
receiving information relating to the flow of electrical current across a surface of biological tissue, the flow defining an upstream direction and a downstream direction;
ablating the biological tissue to form or modify a bluff body to create a tail portion, the tail portion being aligned so as to lie on the downstream side of the bluff body and pointing away from the direction of the incoming current.

2. The method of claim 1, including the step of forming or modifying the bluff body to create a symmetrical teardrop shape.

3. The method of claim 1, wherein:
the surface of biological tissue is a three-dimensional surface; and
including the step of mapping the surface in two dimensions to orient the bluff body.

4. The method of claim 1, wherein the surface of biological tissue is the inner surface of a human heart.

5. The method of claim 1, including the step of using a thin, triangular heating device to form the bluff body.
